(19) United States
(12) Patent Application Publication
(10) Pub. No.: US 2014/0039573 A1
(43) Pub. Date: Feb. 6, 2014

(54) NEUROMUSCULAR STIMULATOR WITH BATTERY MONITORING, EXTERNAL DATA ADAPTER, AND SIMPLIFIED USER INTERFACE

(71) Applicant: Clifford P. JINDRA, Vancouver, WA (US)

(72) Inventor: Clifford P. JINDRA, Vancouver, WA (US)

(73) Assignee: International Rehabilitative Sciences, Inc., Vancouver, WA (US)

(21) Appl. No.: 13/566,668

(22) Filed: Aug. 3, 2012

(51) Int. Cl. A61N 1/36

(52) U.S. Cl. CPC A61N 1/36003
USPC 607/48

(57) ABSTRACT

A portable electronic neuromuscular stimulator monitors its battery and estimates battery end of life from the number of charging cycles and usage history. The stimulator can also be used with an external adapter to retrieve internally stored data regarding usage. A simplified user interface allows the user to input a level of pain both before and after treatment.

### FIELD OF THE INVENTION

The present invention is directed to a neuromuscular stimulator and more specifically to one that can monitor its battery to determine remaining battery life, that can be used with an external adapter for retrieval of usage data, and that has a simplified user interface to input a pain level.

### DESCRIPTION OF RELATED ART

Neuromuscular stimulators are well known in the art. A battery-powered, portable device is programmed to output pulses through output channels. The channels are connected to electrodes, which the patient places in locations appropriate to the type of muscle stimulation to be applied. The device has a display screen to show a status to the patient and switches to allow the patient to start and, on some devices, select the treatment.

A typical stimulator is disclosed in U.S. Pat. No. 6,393,328, whose disclosure is hereby incorporated by reference in its entirety into the present disclosure. One exemplary embodiment of the multi-functional electro-medical device in accordance with that patent is programmed to apply interferential current stimulation, high voltage muscle stimulation, and pulsed muscle stimulation treatments. With the ability to provide interferential current stimulation, the multi-functional portable electro-medical device provides the ability to treat painful muscle conditions. The multi-functional portable electro-medical device invention may be programmed to apply many other types of electro-medical treatment such as NEMS, TENS, microcurrent, high voltage, constant voltage or pulse width, and the like. The device also has a removable storage card to store data concerning the operation of the device. The storage card can be given to the physician or mailed to a service bureau.

The device of the '328 patent will be described in further detail with reference to FIGS. 1-5 of that patent, which are reproduced herein. FIGS. 1-4 illustrate an exemplary embodiment of the multi-functional portable electro-medical device 10 of the '328 patent. The multi-functional portable electro-medical device 10 includes a power switch 12, a liquid crystal display (LCD) touch screen 14 and a speaker 26. Each of the above-described components, as well as other components to be described later herein, may be housed within a plastic case or shell 24.

As shown in FIGS. 3 and 4, the case or shell 24 of the exemplary electro-medical device 10 may be formed from an upper piece 24a and a lower piece 24b, in order to more easily manufacture the electro-medical device 10. Four output jacks 16-22 may be provided at the rear of the case of the multi-functional portable electro-medical device 10. The four output jacks 16-22 provide a separate jack for each of the output channels. A jack 28 for connecting the electro-medical device 10 to a battery charger (not shown) may be located on, for example, the rear of the electro-medical device 10.

The electro-medical device 10 may be used in a self-administered manner by patients for providing treatments prescribed by physicians and/or other health care providers. A multi-functional portable electro-medical device in accordance with the present invention may be used for any number of muscle treatments including, without limitation: the relaxation of muscle spasms, the prevention or retardation of muscle disuse atrophy, increasing local blood circulation in the legs or other limbs of the patient, reeducating the leg muscles or other muscles of the patient, providing immediate post-surgical stimulation of calf muscles of the patient in order to prevent venous thrombosis, maintaining or increasing the range of motions of the patient's legs or other limbs, relieving acute pain, the relief and management of chronic pain and for reducing edema and/or inflamation as well as many other treatments.

In order to connect the output jacks 16-22 of the electro-medical device 10 to the patient, a like plurality of cables (not shown) is used to make a connection between one of the output jacks and a standard electrode pad (not shown) which contacts the skin of the patient. For safety purposes, a pin of the cable is inserted into each of the respective jacks 16-22 in order to connect an electrode pad to the respective output jack 16-22.

The exemplary embodiment of the multi-functional portable electro-medical device 10 of the '328 patent is a digital device which provides additional safety features for the user. The electro-medical device 10 provides four isolated channels capable of independently treating four separate muscle groups. Each of the four channels has independent output power stages and transformers in order to provide channel separation. The electro-medical device 10 is battery powered in order to provide portability. The battery power of the exemplary embodiment is provided by an internal 7.2 volt nickel cadmium or nickel metal hydride battery system, which eliminates the need for patients to monitor and replace batteries. The LCD touch screen 14 provides visual feedback and an interface for the user. In addition, the circuitry of the electro-medical device 10 includes a speaker 26 that provides audible reinforcement of keystroke actions. Also, each of the electrically isolated channels has a separate intensity control for independently increasing and decreasing the intensity of that channel.

The power switch 12, in addition to powering on the electro-medical device 10, also serves as an off switch for shutting down the device. The muscle stimulation mode contract time and relax time, treatment time and normal/alternating mode selections have built-in default settings. The inferential mode, continuous/variable mode selection, frequency setting, pad selection, and treatment times also have default settings. However, those default settings are easily modified at the time of use in accordance with the prescription or the user's physician's instructions.

An exemplary embodiment of the electro-medical device 10 of the '328 patent may be provided with a data storage card 30, the details of which are more fully shown and described in U.S. Pat. No. 5,755,745, which is incorporated by reference herein in its entirety. The structure of the storage card 30 is such that it is designed to be used with and removed by the patient from the electro-medical device 10, or any other similar type of Class II device which a patient uses in an unsupervised manner, mailed to a service bureau for downloading the stored usage information, and replaced with a new data storage card. Typically, a data storage card such as the data storage card 30 disclosed herein is designed to hold 30-60 days of patient usage information, although more modern stimulators can store up to two years of data. During treatment use by the patient, data is accumulated for the treatment period on the data storage card 30.

FIG. 5 is a schematic block diagram of an exemplary embodiment of a multi-functional portable electro-medical device 10 of '328 patent. The exemplary electro-medical device 10, as previously discussed, is powered by a rechargeable 7.2 volt nickel cadmium or nickel hydride battery system 36, which is recharged, by a battery charger 38, which may preferably be powered by standard 110 volt household electric current. As a safety feature, the electro-medical device 10 is designed to be inoperative while the battery system 36 is being charged. A battery monitor circuit 40 is connected between the battery system 36 and the processor 42 to control battery charging. The processor 42 serves to control and monitor all of the functions of the electro-medical device 10.

However, the fact that the device is battery-powered means that the battery will eventually reach the end of its useful life and need to be replaced. While the device of the '328 patent has a battery monitor circuit 40, that battery monitor circuit simply determines the amount of charge needed during a charging operation.

Also, retrieval of usage data is not convenient. Once the storage card is removed, there must be some way to read it, and not all computers will have compatible card readers.

Furthermore, known devices do not provide user-friendly ways of allowing a user to select the level of treatment and do not accept inputs to assess the success of the treatment.

### SUMMARY OF THE INVENTION

A need therefore exists in the art to improve the above matters.

It is therefore an object of the invention in at least some embodiments to estimate battery end-of-life time.

It is another object of the invention in at least some embodiments to provide a more convenient way to retrieve stored usage information.

It is yet another object of the invention to provide an easy way to let the patient select the level of treatment.

It is still another object of the invention to collect data from which the effectiveness of the treatment can be assessed.

To achieve the above and other objects, the present invention in at least one embodiment predicts the battery end of life by using the accumulated data stored in the battery data registers, specifically the age of the battery pack and the number of used charging cycles, and the patient usage data.

The present invention in at least one other embodiment has a data port for connection to an external adapter. The external adapter can have its own nonvolatile memory or can connect to a conventional memory device such as a USB flash drive.

The present invention in at least one other embodiment has a simplified interface to allow the user to rate the pain level. If the user does so before the treatment, the level of treatment can be automatically determined accordingly. If the user does so both before and after the treatment, the data can be saved to allow a physician or other party to determine the effectiveness of the treatment.

The present invention can use any or all features of the stimulator of the '328 patent or of any other stimulator. Also, the battery monitoring technique of the present invention can be used in devices other than neuromuscular stimulators, i.e., any device that utilizes a batter that can be monitored as described above.

### BRIEF DESCRIPTION OF THE DRAWINGS

Preferred embodiments of the present invention will be set forth in detail with reference to the drawings, in which:

FIG. 1 is a perspective view of an exemplary embodiment of a multi-functional portable electro-medical device in accordance with the '328 patent;

FIG. 2 is a top view of the portable electro-medical device of FIG. 1;

FIG. 3 is a front elevation view of the portable electro-medical device of FIG. 1;

FIG. 4 is a rear elevation view of the multi-functional portable electro-medical device of FIG. 1;

FIG. 5 is a schematic block diagram of an exemplary multi-functional portable electro-medical device in accordance with the '328 patent;

FIG. 6 is a top view of a stimulator according to any of the preferred embodiments;

FIG. 7 is a side view of the stimulator of FIG. 6;

FIG. 8 is a top perspective view of the stimulator of FIG. 6;

FIG. 9 is a bottom perspective view of the stimulator of FIG. 6;

FIG. 10 is a flow chart showing calculation triggering in the first preferred embodiment;

FIG. 11 is a flow chart showing the calculation of the estimated battery end of life in the first preferred embodiment;

FIG. 12 is a perspective view showing the use of an adapter according to the second preferred embodiment; and

FIG. 13 is a top view of a stimulator having a user interface according to the third preferred embodiment.

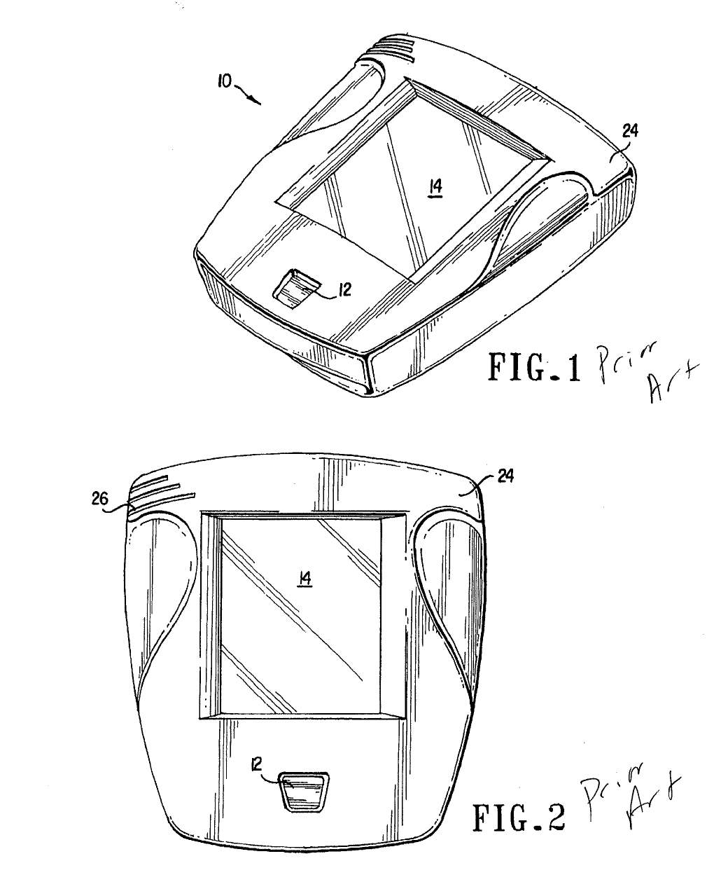

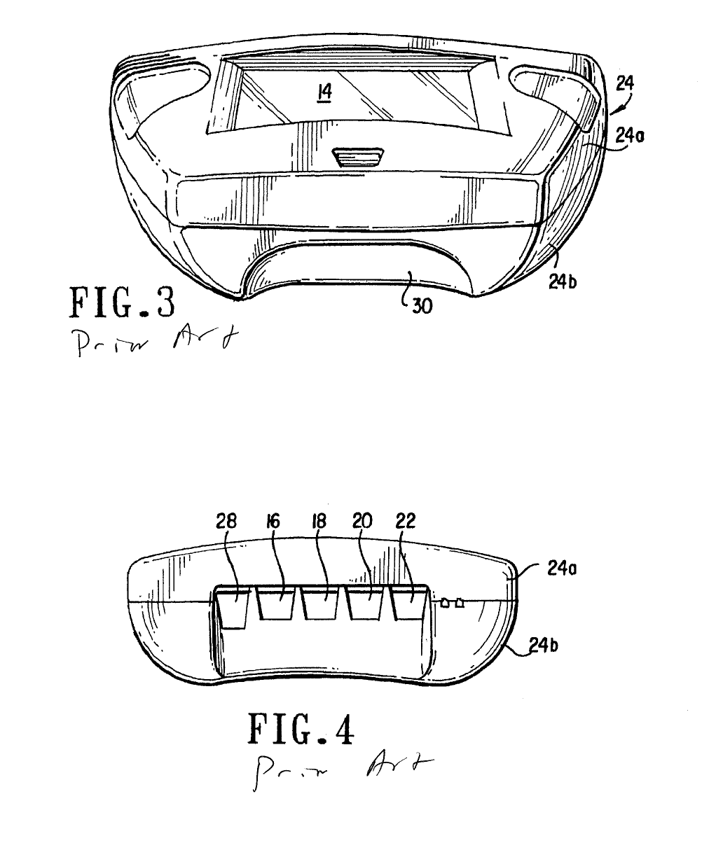

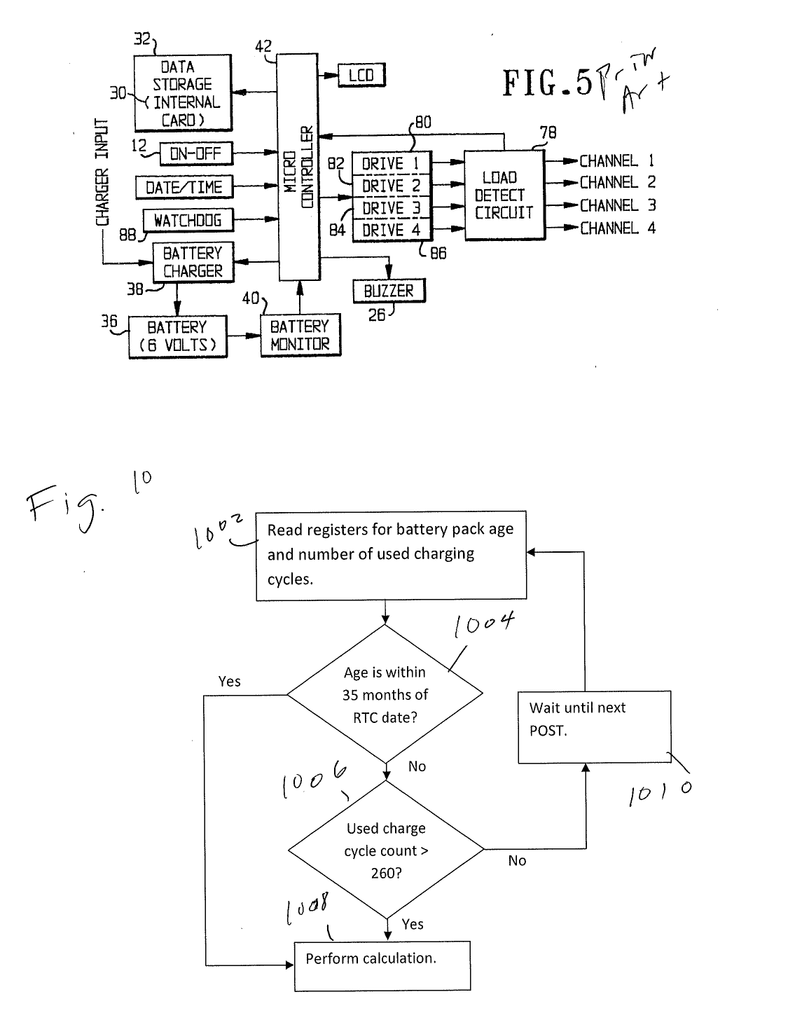

### DETAILED DESCRIPTION OF THE PREFERRED EMBODIMENTS

Preferred embodiments of the present invention will be set forth in detail with reference to the drawings, in which like reference numerals refer to like elements or steps throughout.

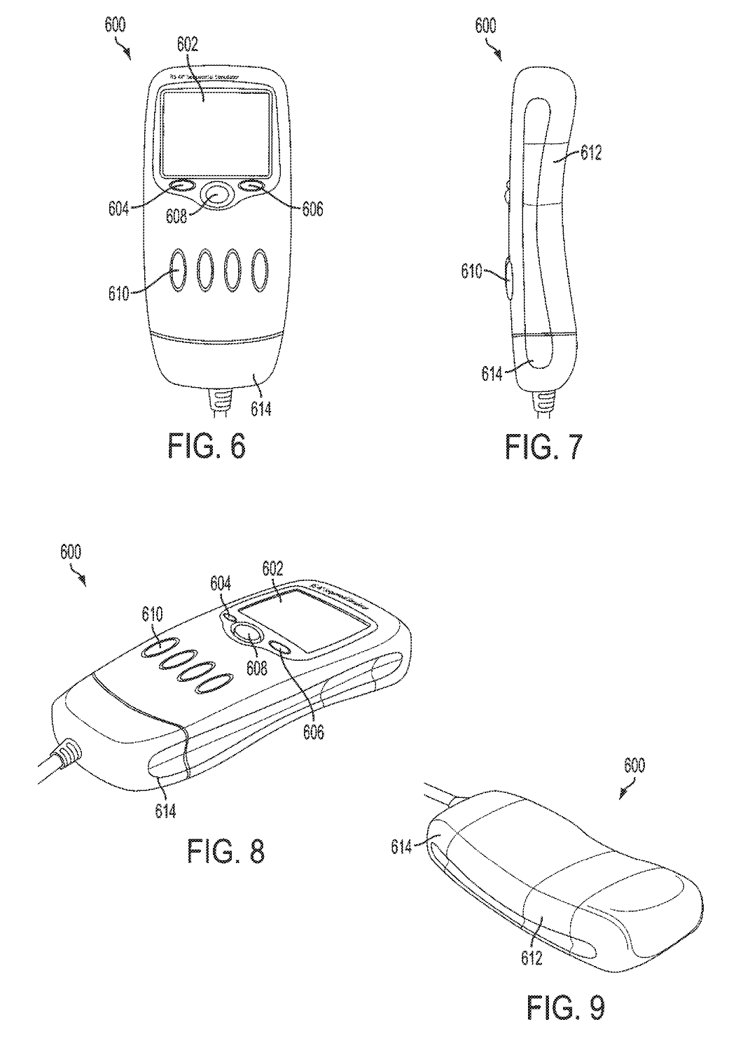

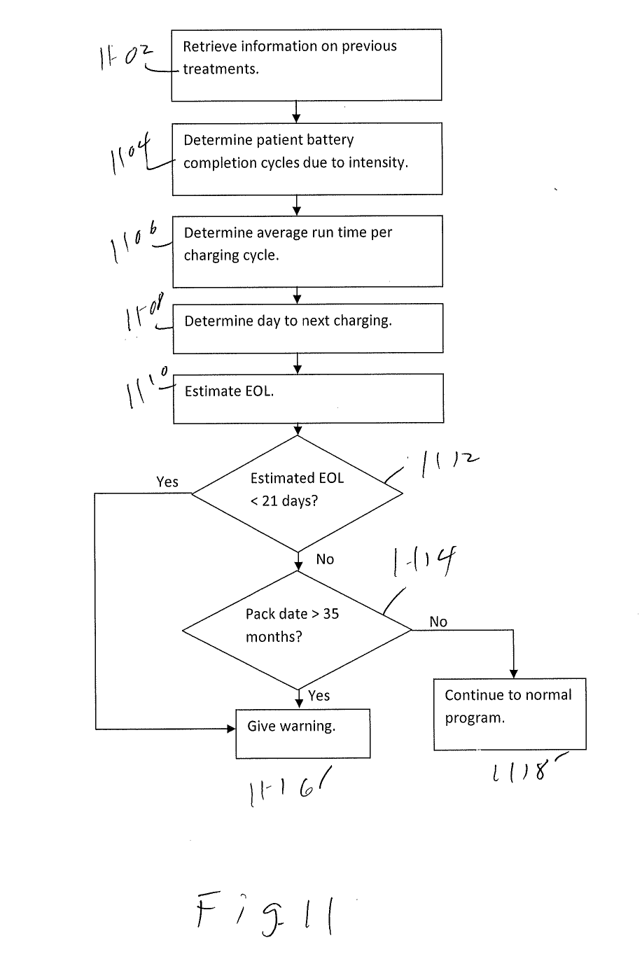

FIGS. 6-9 show a top view, a side view, a top perspective view, and a bottom perspective view, respectively, of a stimulator 600 according to the preferred embodiments. The stimulator 600 can incorporate any or all features of the '328 stimulator or any other stimulator. The stimulator 600 includes a display screen 602, a left function key 604, a right function key 606, an on/off/pause key 608, four intensity keys (one for each channel) 610, a lithium-ion (or other suitable) battery 612, and a connector 614.

The first preferred embodiment concerns battery monitoring. In order to provide the patient with adequate notice that the main battery is approaching its end of life and requires replacement, the device uses the accumulated data stored in the battery data registers and the patient usage data stored in the device NVRAM to predict when a battery will reach its end of life status. First, the device determines whether the prediction is needed. That determination, called calculation triggering, will be explained with reference to the flow chart of FIG. 10.

At POST (power on self test), the battery registers for the battery age (pack date) and the number of used charging cycles are read (step 1002). Predictive calculations begin when the battery age (pack date) is 35 months prior to the RTC (real-time clock) date (step 1004) or the used charge cycle count is greater than 260 (step 1006). In either of those cases, the device performs the calculation (step 1008) in the manner explained below. When the battery age is less than 35 months and the charge cycle count is less than 260, no calculation will be done. Instead, the device will wait until the next POST (step 1010) to do the determination again.

The calculation is performed in the following manner, which will be explained with reference to the flow chart of FIG. 11. The following empirical data are used: At 100% intensity, the normal INF depletion time is 100 minutes, while the normal NMES depletion time is 524 minutes

### DEFINITION OF TERMS

Average Number of Treatments per Day: The average number of INF treatments per day is determined by adding the number of INF treatment segments within the timeframe of evaluation (which is typically the last 20 days) and dividing the sum by the number of days in that time frame of evaluation. Similarly, the average number of NMES treatments per day is determined by adding the number of NMES treatment segments within the timeframe of evaluation and dividing the sum by the number of days in the time frame of evaluation.

Average Treatment Times: The average INF treatment time is determined by summing the times of all of the INF treatments and dividing that sum by the number of INF treatments. Similarly, the average NMES time is determined by summing the times of all of the NMES treatments and dividing that sum by the number of NMES treatments.

Average Intensities: The average INF intensity is determined by summing all of the INF intensity settings within the timeframe and dividing the sum by 4 times the number of INF treatments within the timeframe. Similarly, the average NMES intensity is determined by summing all NMES intensity settings within the timeframe and dividing the sum by 4 times the number of NMES treatments within the timeframe.

When the calculation is triggered (FIG. 10, step 1008), the device MCU evaluates the patient usage data from the last 20 days stored in the NVRAM for the following information (refer to the definitions of terms above) (FIG. 11, step 1102):

Average INF Intensity

Average NMES Intensity

Average Number of Treatments per Day

Average INF Treatment Time

Average NMES Treatment Time

Patient Battery Depletion Cycles Due to Intensity (step 1104): For INF treatments, that quantity, referred to as NormINFDepletionTime, is calculated as 100×(100/Average INF Intensity). For NMES treatments, that quantity, referred to as NormNMESDepletionTime, is calculated as 524×(100/Average NMES Intensity).

Average Run Time per Charging Cycle (step 1106): That quantity, referred to as EstimatedTreatments, is calculated from the quantities calculated above as [(NormINFDepletionTime/Average INF Treatment Time)+(NormNMESDepletionTime/Average NMES Treatment Time)].

Day to next charging (step 1108): That quantity, referred to as DayToNextCharging, is calculated as EstimatedTreatments/Average Number of Treatments per Day.

Estimated EOL (step 1110)=(300−Cycle Count)/DayToNextCharging).

The processor determines whether to give a warning message in the following manner. If the estimated EOL is less than 21 Days (step 1112) or the pack date is greater than 35 months before the RTC date, (step 1114), the device a “Replace battery” message (step 1116) for 10 seconds with the backlight on and sounds the alert beep three times. A patient data record for treatment is written with the timestamp as evidence that the message was displayed to the patient. If the estimated EOL is greater than 20 days and the pack date is <35 months, the normal program is continued (step 1118).

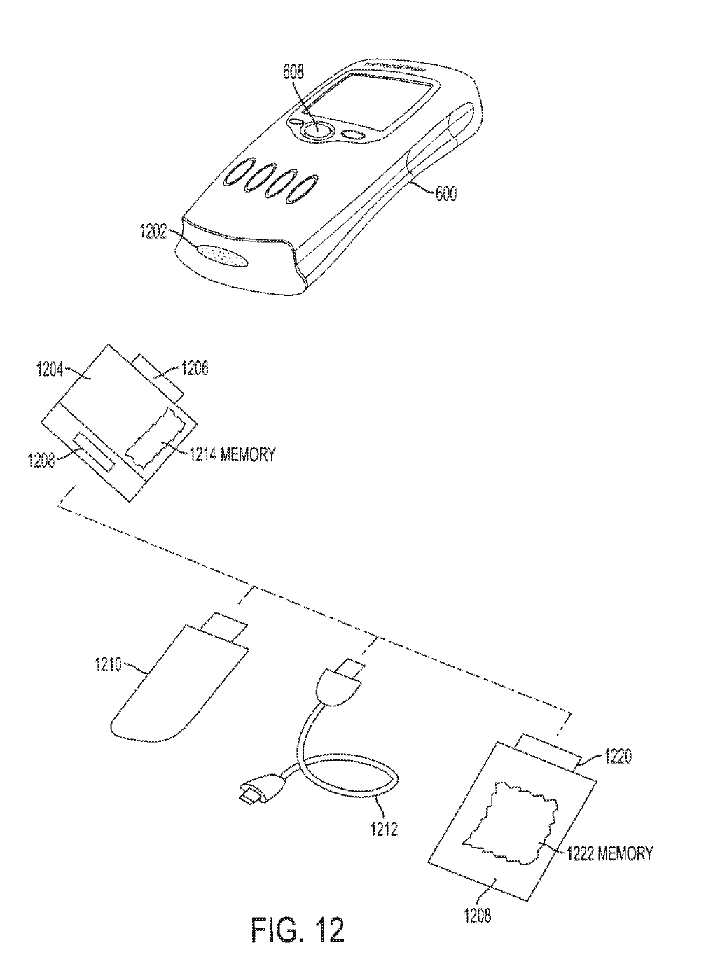

Another embodiment uses an adapter to allow downloading of patient data. That embodiment will be disclosed with reference to FIG. 12. As shown in that figure, a stimulator 600 has had its connector 614 removed to reveal a DB-25 (or other suitable) port 1202. A patient data download adapter 1204 has a DB-25 connector 1206 to connect to the DB-25 port 1202 on the stimulator 600. The adapter 1204 also has a USB 2.0 (or other suitable) port 1208 to connect to a known USB flash drive 1210, a known USB cable 1212, or another USB device. The adapter 1204 also has a non-volatile memory 1214. The specifics of the DB-25 and USB pin assignments are known in the art and will therefore not be described in detail here. USB has been selected because of the high likelihood of compatibility with existing thumb drives and computers, although any other standard could be used.

Patient data download adapters are identified using the DB-25 pins labeled DETECT1 and DETECT2 set to bx01. The device 600 will then access the memory location named “Transfer Type” within the adapter's memory 1214. The “Transfer Type” HEX Data Byte will identify the adapter type according to the following table:

TABLE I

| HEX Data Byte | Adapter Type |
|---|---|
| 00 | Not to be used |
| 01 | USB |
| 02 | NVRAM |
| 03-FF | Reserved for future types |

Since technology continues to change, the reserved HEX data bytes (03-FF) will be used to identify future adapters to take advantage of the most current technology available. Patient data download will be done in the prescribing physician's office by the physician, a member of the physician's staff, a sales representative, or a case manager. The physician will insert a flash drive 1210 into the USB 2.0 port built into the adapter 1204 and turn the device on using the On/Off/Pause key 608. The device 600 detects the presence of a patient data download adapter 1204 using the DETECT1 and DETECT2 pins on the DB-25 connector 1206, identifies the type as USB using the “Transfer Type” HEX data byte, and begins a routine to download the header information (serial number, etc.) and up to two years of patient data saved on the device. Upon completion of the download, the device 600 will beep using the treatment-completed sound and then turn off. When the flash drive 1210 is removed from the device 600 and inserted into the physician's PC (not shown), a program provided on a CD or other medium or downloaded from a server on the Internet will retrieve the data from the flash drive 1210 and format it to produce a patient report for the previous period of usage.

In rare cases, the patient will need to be provided with a special adapter 1218 having a DB-25 connector 1220 mating to the DB-25 connector 1202 of the device 600. The adapter 1218 will have a self-contained non-volatile memory 1222 of sufficient size to hold the two years of patient data contained in the device memory. The device 600 detects the presence of a patient data download adapter 1218 using the DETECT1 and DETECT2 pins on the DB-25 connector, identifies the type as a NVRAM using the “Transfer Type” HEX data byte in the manner described above, and begins a routine to download the header information (serial number, etc.) and up to the two years of patient data saved on the device 600 into the memory 1222 of the adapter 1218. Upon completion of the download, the device 600 will beep using the treatment-completed sound and then turn off. The patient will then return the adapter 1218 to the company providing the device 600, where the full two years of patient usage data will be retrieved from the memory 1222 in the adapter 1218 and formatted.

Alternatively, either the physician or the patient can connect the device 600 to a computer (not shown) via the adapter 1204 and a USB cable 1212 to transfer data to the computer using software provided by the vendor of the device 600 and installed on the computer. In the case of a patient, the patient can be given instructions to connect via the computer to a secure Web site to transfer the information to a remote site.

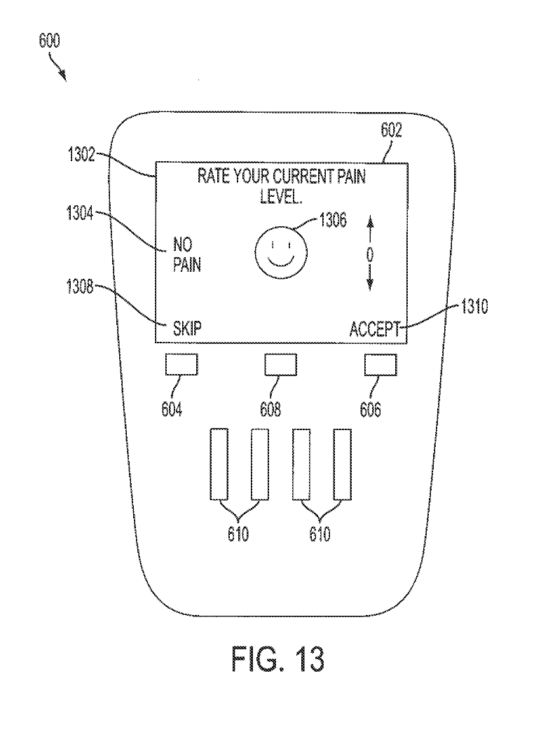

A third preferred embodiment, which will be explained with reference to FIG. 13, offers a simplified user interface. The stimulator 600, as noted above, has four up-down keys 610, one to control each channel, and a display 602. The device 600 outputs a prompt 1302 for the user to select the level of pain. Using any of the keys 610, the user can select a pain level from 0 (no pain) to 10 (hurts worst). For the currently selected pain level, the device 600 displays at least one of a text description (“No pain”) 1304 and a graphic depiction (emoticon with appropriate facial expression) 1306. The display also provides prompts 1308, 1310 to skip the step and to select the currently selected pain level, which may be selected with the keys 604 and 606, respectively. The user can make the selection before and after treatment to select an appropriate level of treatment and to provide an input of the effectiveness of the treatment. In a device combining the second and third preferred embodiment, the “before” and “after” data can be output through the adapter.

While preferred embodiments of the invention have been set forth above, those skilled in the art who have reviewed the present disclosure will readily appreciate that other embodiments can be realized within the scope of the invention. For example, numerical values and disclosures of specific technologies are illustrative rather than limiting. Also, the various preferred embodiments and their modifications can be used together or separately. Therefore, the present invention should be construed as limited only by the appended claims.

What is claimed is:

1. A device comprising:
a battery;
a processor in communication with the battery; and
a user interface in communication with the processor;
wherein the processor is configured to receive inputs of a number of charge cycles of the battery and a usage history of the device and to estimate an end-of-life time of the battery in accordance with the number of charge cycles and the usage history, and to provide an output through the user interface in accordance with the estimated end-of-life time of the battery.

2. The device of claim 1, wherein the processor is configured to provide the output when the end-of-life time of the battery is less than a predetermined time.

3. The device of claim 1, wherein the user interface comprises a display.

4. The device of claim 1, wherein the user interface comprises an audible interface.

5. The device of claim 1, wherein the device is a neuromuscular stimulator.

6. The device of claim 6, wherein the usage history comprises information concerning a number of treatments, a length of each treatment, and an intensity of each treatment.

7. A method for estimating an end-of-life time of a battery in a device having a processor and a user interface, the method comprising:
(a) receiving inputs of a number of charge cycles of the battery and a usage history of the device into the processor;
(b) automatically estimating, in the processor, the end-of-life time of the battery in accordance with the number of charge cycles and the usage history; and
(c) automatically providing an output through the user interface in accordance with the estimated end-of-life time of the battery.

8. The method of claim 7, wherein step (c) is performed when the end-of-life time of the battery is less than a predetermined time.

9. The method of claim 7, wherein the user interface comprises a display.

10. The method of claim 7, wherein the user interface comprises an audible interface.

11. The method of claim 7, wherein the device is a neuromuscular stimulator.

12. The method of claim 11, wherein the usage history comprises information concerning a number of treatments, a length of each treatment, and an intensity of each treatment.

13. A system comprising:
an electronic neuromuscular stimulator having a processor, an internal memory, and a data port; and
an adapter configured to be connected to the data port of the stimulator to receive data from the internal memory of the stimulator;
wherein the processor of the stimulator is configured to detect a presence of the adapter when the adapter is connected to the data port, to detect a type of data transfer required by the adapter, and to output the data through the data port to the adapter.

14. The system of claim 13, wherein the adapter comprises a port for connection to an external memory device and is configured to pass the data to the external memory device.

15. The system of claim 14, wherein the adapter comprises an internal non-volatile memory and is configured to store the data in the non-volatile memory of the adapter.

16. A method for exporting data from an electronic neuromuscular stimulator having a processor, an internal memory, and a data port, the method comprising:
(a) providing an adapter configured to be connected to the data port of the stimulator to receive data from the internal memory of the stimulator;
(b) connecting the adapter to the data port;
(c) automatically detecting a presence of the adapter when the adapter is connected to the data port;
(d) automatically detecting a type of data transfer required by the adapter; and
(e) automatically outputting the data through the data port to the adapter.

17. The method of claim 16, wherein the adapter comprises a port for connection to an external memory device and is configured to pass the data to the external memory device, and wherein the method further comprises (f) storing the data in the external memory device.

18. The method of claim 16, wherein the adapter comprises an internal non-volatile memory and is configured to store the data in the non-volatile memory, and wherein the method further comprises (f) storing the data in the non-volatile memory of the adapter.

19. A portable electronic neuromuscular stimulator comprising:
a processor for controlling the stimulator;
electronics for outputting stimulation signals under control of the processor; and
a user interface, under control of the processor, for receiving from a user an indication of a level of pain, the user interface comprising at least one key for allowing the user to enter a numeric indication of the level of pain

20. The stimulator of claim 19, wherein the processor is configured to control the user interface to prompt the user for the indication of the level of pain before and after treatment.

21. A method for supplying treatment using a portable electronic neuromuscular stimulator having a user interface with at least one key for allowing a user to enter a numeric value, the method comprising:
(a) prompting the user to enter the numeric value to indicate a level of pain; and
(b) applying treatment in accordance with the level of pain.

22. The method of claim 21, further comprising (c) prompting the user to enter the numeric value again after the treatment of step (b).
